(19) United States
(12) Patent Application Publication
(10) Pub. No.: US 2021/0275362 A1
(43) Pub. Date: Sep. 9, 2021

(54) METHODS AND APPARATUSES FOR MAKING ELASTOMERIC LAMINATES WITH ELASTIC STRANDS UNWOUND FROM INDIVIDUAL SPOOLS

(71) Applicant: The Procter & Gamble Company, Cincinnati, OH (US)

(72) Inventors: Uwe SCHNEIDER, Cincinnati, OH (US); Joseph Allen ECKSTEIN, Sunman, IN (US); Masaharu NISHIKAWA, Cincinnati, OH (US); Randall Allen MYERS, Fairfield, OH (US); Tina BROWN, Cincinnati, OH (US)

(21) Appl. No.: 17/189,476

(22) Filed: Mar. 2, 2021

Related U.S. Application Data
(60) Provisional application No. 62/984,837, filed on Mar. 4, 2020.

(51) Int. Cl. A61F 13/15

(52) U.S. Cl. CPC A61F 13/15764; A61F 2013/1591; A61F 13/15739; A61F 13/15772

(57) ABSTRACT

The present disclosure relates to methods for making elastomeric laminates that may be used as components of absorbent articles. During assembly of the elastomeric laminate, elastic material may be advanced and stretched in a machine direction and joined with either or both first and second substrates advancing in the machine direction. The apparatuses according to the present disclosure may be configured with a plurality of spools, wherein each spool comprises a single elastic strand wound onto a core. The elastic strands are unwound from respective spools by rotating the spools about the cores. Neighboring elastic strands may be spaced or separated from each other at a desired distance in a cross direction by advancing the elastic strands through a strand guide that may comprise a plurality of tines or reeds. The assembled elastomeric laminate may then be accumulated by being wound onto a roll or festooned in a container.

### CROSS REFERENCE TO RELATED APPLICATION

This application claims the benefit of U.S. Provisional Application No. 62/984,837, filed Mar. 4, 2020, which is incorporated herein by reference.

### FIELD OF THE INVENTION

The present disclosure relates to methods for manufacturing absorbent articles, and more particularly, to apparatuses and methods for making elastomeric laminates that may be used as components of absorbent articles.

### BACKGROUND OF THE INVENTION

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from an advancing web or webs are combined with other individual components created from other advancing webs. Webs of material and component parts used to manufacture diapers may include: backsheets, topsheets, leg cuffs, waist bands, absorbent core components, front and/or back ears, fastening components, and various types of elastic webs and components such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

Some absorbent articles have components that include elastomeric laminates. Such elastomeric laminates may include an elastic material bonded to one or more nonwovens. The elastic material may include an elastic film and/or elastic strands. In some laminates, a plurality of elastic strands are joined to a substrate, such as a nonwoven, while the plurality of strands are in a stretched condition so that when the elastic strands relax, the nonwoven gathers between the locations where the nonwoven is bonded to the elastic strands, and in turn, forms corrugations. The resulting elastomeric laminate is stretchable to the extent that the corrugations allow the elastic strands to elongate.

In some assembly processes, stretched elastic strands may be advanced in a machine direction and adhered between two advancing substrates, wherein the stretched elastic strands are spaced apart from each other in a cross direction. Some assembly processes may also be configured to utilize relatively large numbers of individual elastic strands having relatively low decitex with the elastic strands being very closely spaced apart from each other in a cross direction. In some configurations, close cross directional spacing between low decitex elastic strands can be achieved by drawing such elastic strands that have been previously been wound onto a beam. Figure A shows an example beam 50 that may include two side plates 51 connected with opposing ends of a mandrel core 52, and Figure B shows an example of the beam of Figure A with a plurality of strands 52 wound thereon.

When assembling beams of elastic strands, relatively low decitex individual elastic strands unwound from respective spools may be wound onto a beam. While advancing from the individual spools, the elastic strands are closely spaced from each before being wound side by side onto the beam. In some configurations, elastic strands may have decitex values that are below 500, and as such, relatively large numbers of elastic strands, for example hundreds, of individual elastic strands may be wound onto a single beam with relatively close cross directional spacing. It is to be appreciated that winding beams with relatively large numbers of elastic strands drawn from individual spools may require relatively large assembly areas to accommodate correspondingly large numbers of spools. In addition, the low decitex values and large numbers of elastic strands may result in a relatively delicate assembly process that may require close monitoring and control to help ensure that strands are not broken while being wound onto the beam. Once the elastic strands are wound onto the beams of elastic strands, the beams may be transported to a location wherein the elastic strands are unwound from the beams and used with elastic laminate assembly processes. However, problems can be encountered with manufacturing processes involving the construction of elastic laminates with elastic strands drawn from beams.

For example, relatively low decitex elastic strands may be coated with a spin finish before being wound onto individual spools. In some configurations, relatively low decitex elastic strands may be unwound from the spools and then coated with a spin finish before being wound onto the beams. The spin finish, sometimes referred to a yarn finish, is a coating that helps prevent the elastics strands from adhering to themselves, each other, and/or downstream handling equipment. When constructing absorbent articles, hot melt adhesives are sometimes used to adhere stretched elastic strands to advancing substrates to create elastic laminates. However, hot melt adhesives used to adhere stretched elastic strands to substrates when constructing absorbent articles may not adhere well to strands having a spin finish. As such, increased amounts of adhesive may be required to adequately adhere the stretched elastic strands to the substrates than would otherwise be required for elastic strands without a spin finish. In turn, relatively larger amounts of adhesives required to bond the elastic strands to the substrates may have a negative impact on aspects of the resulting product, such as with respect to costs, functionality, and aesthetics.

Similar to the beam winding process, unwinding elastic strands from a beam may also be a relatively delicate assembly process that may require close monitoring and control to help ensure that strands are not broken while being incorporated into an elastic laminate assembly process. For example, one broken elastic strand during the beam unwinding process can have a relatively large negative impact on the assembly process as a whole. When utilizing beams, several elastic strands are unwound in close proximity to one another. Thus, a violent and uncontrolled retraction of a loose end of a broken strand under tension may also cause additional strands to become broken. In addition, during the unwinding process, a winding of a broken elastic strand on a beam may eventually collapse onto neighboring windings of elastic strands that continue to be unwound from the beam, thus potentially causing additional elastic strand breaks. As such, in some configurations, an entire manufacturing line may need to be temporarily stopped while the defective beam is replaced. Manufacturing lines in the textile industry often operate at relatively slow speeds, and as such, these textile manufacturing lines can be temporarily stopped to replace a defective beam and may not result in a major disruption to production. However, some manufacturing lines, such as disposable absorbent article manufacturing lines, may operate at relatively high speeds that may exacerbate problems associated with strand breakouts, necessitating beams of elastics to be replaced relatively often. As such, it can be inefficient and/or cost prohibitive to frequently stop and restart high speed manufacturing operations to replace beams.

In some configurations, it may be desirable to have elastic strands joined between to substrates such that resulting the elastomeric laminate may have different stretch characteristics in different regions along the laminate width or cross direction CD. In turn, when the elastomeric laminate is elongated, some elastic strands may exert contraction forces that are different from contraction forces exerted by other elastic strands. Such differential stretch characteristics can be achieved by stretching some elastic strands more or less than other elastic strands before joining the elastic strands with the substrates. However, stretching some elastic strands more less than others may be difficult when drawing relatively closely spaced, low decitex elastic strands from a beam.

Consequently, it would be beneficial to provide a method and apparatus for producing elastomeric laminates with relatively large numbers of closely spaced, low decitex elastic strands without having to first wind the strands onto beams; and/or without the need to coat the strands with spin finish and/or reducing the amounts of spin finish on the strands, while at the same time mitigating negative effects associated with strand breakouts.

### SUMMARY OF THE INVENTION

In one form, a method for assembling an elastomeric laminate comprises steps of: providing first spools, each first spool comprising a single first elastic strand; unwinding the first elastic strands from the first spools; spacing neighboring first elastic strands at a first distance from each other in a cross direction by advancing the first elastic strands in a machine direction through a strand guide; stretching the first elastic strands in the machine direction; combining the first elastic strands with a first substrate and a second substrate to form an elastomeric laminate; and accumulating the elastomeric laminate.

In another form, a method for assembling an elastomeric laminate comprises steps of: providing first spools, each first spool comprising a single first elastic strand; providing second spools, each second spool comprising a single second elastic strand; unwinding first elastic strands from first spools and unwinding second elastic strands from second spools; stretching the first and second elastic strands in the machine direction, wherein the first elastic strands are stretched more than the second elastic strands by rotating first spools and the second spools at different speeds; spacing neighboring first elastic strands at a first distance from each other in a cross direction by advancing the first elastic strands through reeds; spacing neighboring second elastic strands at a second distance from each other in the cross direction by advancing the second elastic strands through reeds; combining the first and second elastic strands with a first substrate and a second substrate to form an elastomeric laminate; and accumulating the elastomeric laminate.

In yet another form, a method for assembling an elastomeric laminate comprises steps of: providing first spools, each first spool comprising a single first elastic strand, wherein the first elastic strands comprise a first decitex; providing second spools, each second spool comprising a single second elastic strand, wherein the second elastic strands comprise a second decitex that is not equal to the first decitex; unwinding first elastic strands from first spools and unwinding second elastic strands from second spools by rotating the first and second spools; stretching the first and second elastic strands in a machine direction; spacing neighboring first elastic strands at a first distance from each other in a cross direction; spacing neighboring second elastic strands at a second distance from each other in the cross direction; combining the first and second elastic strands with a first substrate and a second substrate to form an elastomeric laminate; reducing tension on the elastomeric laminate to allow the stretched first and second elastic strands to contract and form a gathered elastomeric laminate; and accumulating the gathered elastomeric laminate.

In still another form, a method for assembling an elastomeric laminate comprises steps of: providing spools, each spool comprising a single elastic strand; unwinding the elastic strands from the spools by rotating the spools; spacing neighboring elastic strands from each other in a cross direction by advancing the elastic strands in a machine direction through a strand guide; stretching the elastic strands in the machine direction; combining the elastic strands with a first substrate and a second substrate to form an elastomeric laminate; maintaining tension on the elastomeric laminate to prevent the stretched elastic strands from contracting; and accumulating the elastomeric laminate while under tension.

### BRIEF DESCRIPTION OF THE DRAWINGS

Figure A shows an example of an empty beam having two side plates connected with opposing end portions of a mandrel core.

Figure B shows an example of the beam of Figure A with a plurality of strands wound thereon.

FIG. 1A is a front perspective view of a diaper pant.

FIG. 1B is a rear perspective view of a diaper pant.

FIG. 2 is a partially cut away plan view of the diaper pant shown in FIGS. 1A and 1B in a flat, uncontracted state.

FIG. 3A is a cross-sectional view of the diaper pant of FIG. 2 taken along line 3A-3A.

FIG. 3B is a cross-sectional view of the diaper pant of FIG. 2 taken along line 3B-3B.

FIG. 4 is a schematic side view of a converting apparatus adapted to manufacture an elastomeric laminate including a plurality of elastic strands positioned between a first substrate and a second substrate.

FIG. 4A is a schematic side view of a converting apparatus adapted to manufacture an elastomeric laminate that is advanced directly to an absorbent article assembly line.

FIG. 5 is a view of the converting apparatus of FIG. 4 taken along line 5-5.

FIG. 6 is an isometric view of a spool of an elastic strand wound onto a core.

FIG. 7 is a front side view of an unwinder.

FIG. 8 is a front side view of a strand guide.

FIG. 9 is a front side view of a front side view of an unwinder configured as a surface driven unwinder.

FIG. 10 is a view of the unwinder of FIG. 9 taken along line 10-10.

### DETAILED DESCRIPTION OF THE INVENTION

The following term explanations may be useful in understanding the present disclosure: “Absorbent article” is used herein to refer to consumer products whose primary function is to absorb and retain soils and wastes. Absorbent articles can comprise sanitary napkins, tampons, panty liners, interlabial devices, wound dressings, wipes, disposable diapers including taped diapers and diaper pants, inserts for diapers with a reusable outer cover, adult incontinent diapers, adult incontinent pads, and adult incontinent pants. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1, all of which are incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, all of which are incorporated by reference herein.

The present disclosure relates to methods for manufacturing absorbent articles, and in particular, to methods for making elastomeric laminates that may be used as components of absorbent articles. The elastomeric laminates may include a first substrate, a second substrate, and elastic material located between the first substrate and second substrate. During the process of making the elastomeric laminate, the elastic material may be advanced and stretched in a machine direction and may be joined with either or both the first and second substrates advancing in the machine direction. The methods and apparatuses according to the present disclosure may be configured with a plurality of spools, wherein each spool comprises a single elastic strand wound onto a core. The elastic strands are unwound from respective spools by rotating the spools about the cores. Neighboring elastic strands may also be spaced or separated from each other at a desired distance in a cross direction by advancing the elastic strands in a machine direction through a strand guide, such as a comb that may comprise a plurality of tines or reeds. The elastic strands are also stretched in the machine direction and combined with a first substrate and a second substrate to form an elastomeric laminate. Tension on the elastomeric laminate may then be reduced to allow the stretched elastic strands to contract and form a gathered elastomeric laminate. In turn, the gathered elastomeric laminate may be accumulated, such as for example, by being wound onto a roll or being festooned in a container. The accumulated elastomeric laminate may be stored and/or moved to a location for incorporation into a manufacturing process, such as an absorbent article assembly process, wherein the elastomeric laminate may be converted into an absorbent article component.

As discussed in more detail below, the apparatuses herein may be configured to assemble elastomeric laminates with relatively large numbers of closely spaced elastic strands having relatively low decitex values that are unwound from individual spools. As such, it is to be appreciated that the arrangements herein may provide certain advantages over other manufacturing processes utilizing relatively large quantities of elastic strands that are unwound from a beam. For example, utilizing individual elastic strands unwound from individual spools may provide a relatively more robust process that may continue to operate with some quantities of broken elastic strands and may not be subject to relatively frequent line stops due to elastic breakouts that may otherwise occur with beam elastic arrangements. In addition, unwinding individual elastic strands from individual spools may provide relatively more flexibility in creating differential strains in the individual elastic strands and/or spacing between the individual elastic strands as opposed to configurations utilizing large quantities of elastic strands unwound from a beam with fixed spacing between the elastic strands.

As previously mentioned, the elastomeric laminates made according to the processes and apparatuses discussed herein may be used to construct various types of components used in the manufacture of different types of absorbent articles, such as diaper pants and taped diapers. To help provide additional context to the subsequent discussion of the process embodiments, the following provides a general description of absorbent articles in the form of diapers that include components including the elastomeric laminates that may be produced with the methods and apparatuses disclosed herein.

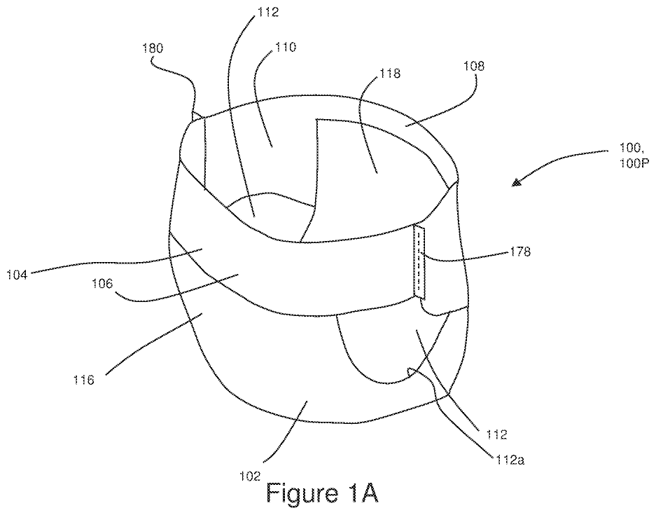

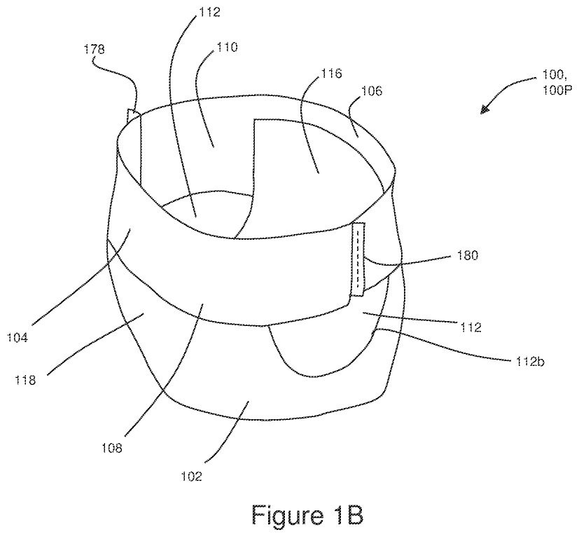

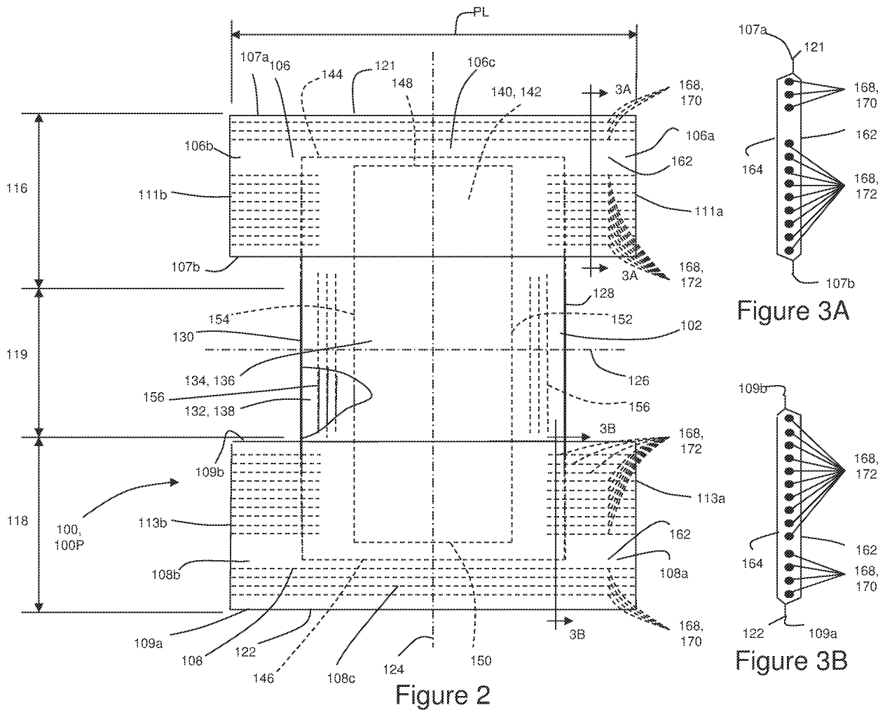

FIGS. 1A, 1B, and 2 show an example of an absorbent article 100 in the form of a diaper pant 100P that may include components constructed from elastomeric laminates assembled in accordance with the apparatuses and methods disclosed herein. In particular, FIGS. 1A and 1B show perspective views of a diaper pant 100P in a pre-fastened configuration, and FIG. 2 shows a plan view of the diaper pant 100P with the portion of the diaper that faces away from a wearer oriented toward the viewer. The diaper pant 100P includes a chassis 102 and a ring-like elastic belt 104. As discussed below in more detail, a first elastic belt 106 and a second elastic belt 108 are bonded together to form the ring-like elastic belt 104.

With continued reference to FIG. 2, the diaper pant 100P and the chassis 102 each include a first waist region 116, a second waist region 118, and a crotch region 119 disposed intermediate the first and second waist regions. The first waist region 116 may be configured as a front waist region, and the second waist region 118 may be configured as back waist region. The diaper 100P may also include a laterally extending front waist edge 121 in the front waist region 116 and a longitudinally opposing and laterally extending back waist edge 122 in the back waist region 118. To provide a frame of reference for the present discussion, the diaper 100P and chassis 102 of FIG. 2 are shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a midpoint of a second longitudinal or left side edge 130 of the chassis 102.

As shown in FIGS. 1A, 1B, and 2, the diaper pant 100P may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100P may also include other features, such as leg elastics and/or leg cuffs to enhance the fit around the legs of the wearer.

As shown in FIG. 2, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 2, the laterally extending end edges 144 and 146 are located longitudinally inward from the laterally extending front waist edge 121 in the front waist region 116 and the laterally extending back waist edge 122 in the back waist region 118. When the diaper pant 100P is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

As previously mentioned, the diaper pant 100P may include a backsheet 136. The backsheet 136 may also define the outer surface 134 of the chassis 102. The backsheet 136 may also comprise a woven or nonwoven material, polymeric films such as thermoplastic films of polyethylene or polypropylene, and/or a multi-layer or composite materials comprising a film and a nonwoven material. The backsheet may also comprise an elastomeric film. An example backsheet 136 may be a polyethylene film having a thickness of from about 0.012 mm (0.5 mils) to about 0.051 mm (2.0 mils). Further, the backsheet 136 may permit vapors to escape from the absorbent core (i.e., the backsheet is breathable) while still preventing exudates from passing through the backsheet 136.

Also described above, the diaper pant 100P may include a topsheet 138. The topsheet 138 may also define all or part of the inner surface 132 of the chassis 102. The topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art. Topsheets 138 may be selected from high loft nonwoven topsheets, apertured film topsheets and apertured nonwoven topsheets. Exemplary apertured films may include those described in U.S. Pat. Nos. 5,628,097; 5,916,661; 6,545,197; and 6,107,539, all of which are incorporated by reference herein.

As mentioned above, the diaper pant 100P may also include an absorbent assembly 140 that is joined to the chassis 102. As shown in FIG. 2, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

As previously mentioned, the diaper 100P may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1, all of which are incorporated by reference herein.

As mentioned above, diaper pants may be manufactured with a ring-like elastic belt 104 and provided to consumers in a configuration wherein the front waist region 116 and the back waist region 118 are connected to each other as packaged, prior to being applied to the wearer. As such, diaper pants may have a continuous perimeter waist opening 110 and continuous perimeter leg openings 112 such as shown in FIGS. 1A and 1B. The ring-like elastic belt may be formed by joining a first elastic belt to a second elastic belt with a permanent side seam or with an openable and reclosable fastening system disposed at or adjacent the laterally opposing sides of the belts.

As previously mentioned, the ring-like elastic belt 104 may be defined by a first elastic belt 106 connected with a second elastic belt 108. As shown in FIG. 2, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. The distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second elastic belt 108. The central region 106c of the first elastic belt is connected with the first waist region 116 of the chassis 102, and the central region 108c of the second elastic belt 108 is connected with the second waist region 118 of the chassis 102. As shown in FIGS. 1A and 1B, the first end region 106a of the first elastic belt 106 is connected with the first end region 108a of the second elastic belt 108 at first side seam 178, and the second end region 106b of the first elastic belt 106 is connected with the second end region 108b of the second elastic belt 108 at second side seam 180 to define the ring-like elastic belt 104 as well as the waist opening 110 and leg openings 112.

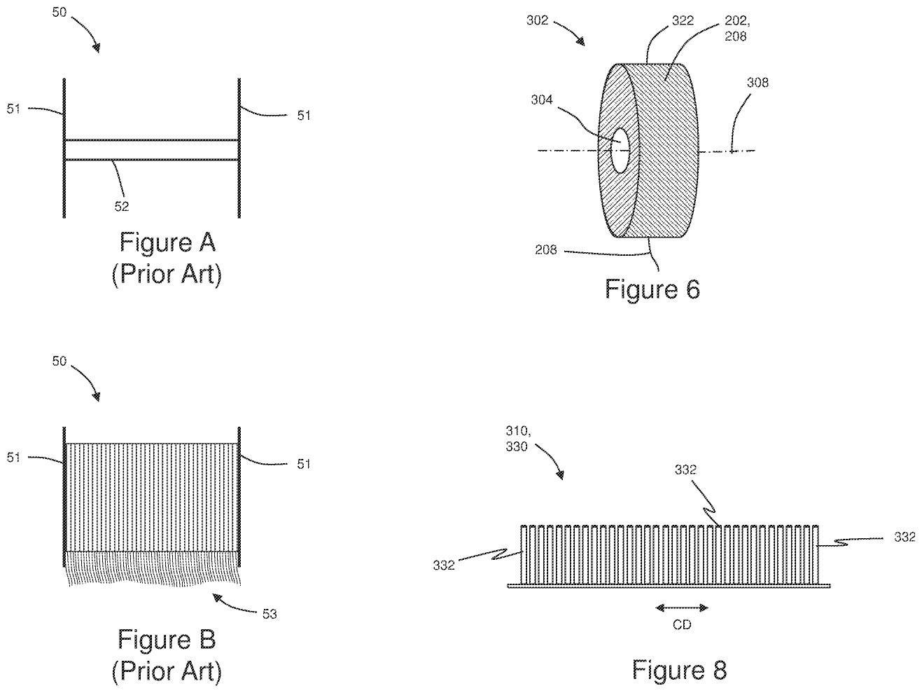

As shown in FIGS. 2, 3A, and 3B, the first elastic belt 106 also defines an outer laterally extending edge 107a and an inner laterally extending edge 107b, and the second elastic belt 108 defines an outer laterally extending edge 109a and an inner laterally extending edge 109b. As such, a perimeter edge 112a of one leg opening may be defined by portions of the inner laterally extending edge 107b of the first elastic belt 106, the inner laterally extending edge 109b of the second elastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100P. The first elastic belt and the second elastic belt may also each include an outer, garment facing layer 162 and an inner, wearer facing layer 164. It is to be appreciated that the first elastic belt 106 and the second elastic belt 108 may comprise the same materials and/or may have the same structure. In some embodiments, the first elastic belt 106 and the second elastic belt may comprise different materials and/or may have different structures. It should also be appreciated that the first elastic belt 106 and the second elastic belt 108 may be constructed from various materials. For example, the first and second belts may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some embodiments, the first and second elastic belts include a nonwoven web of synthetic fibers, and may include a stretchable nonwoven. In other embodiments, the first and second elastic belts include an inner hydrophobic, non-stretchable nonwoven material and an outer hydrophobic, non-stretchable nonwoven material.

The first and second elastic belts 106, 108 may also each include belt elastic material interposed between the outer substrate layer 162 and the inner substrate layer 164. The belt elastic material may include one or more elastic elements such as strands, ribbons, films, or panels extending along the lengths of the elastic belts. As shown in FIGS. 2, 3A, and 3B, the belt elastic material may include a plurality of elastic strands 168 which may be referred to herein as outer, waist elastics 170 and inner, waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. In some embodiments, some elastic strands 168, such as the inner waist elastics 172, may be configured with discontinuities in areas, such as for example, where the first and second elastic belts 106, 108 overlap the absorbent assembly 140. In some embodiments, the elastic strands 168 may be disposed at a constant interval in the longitudinal direction. In other embodiments, the elastic strands 168 may be disposed at different intervals in the longitudinal direction. The belt elastic material in a stretched condition may be interposed and joined between the uncontracted outer layer and the uncontracted inner layer. When the belt elastic material is relaxed, the belt elastic material returns to an unstretched condition and contracts the outer layer and the inner layer. The belt elastic material may provide a desired variation of contraction force in the area of the ring-like elastic belt. It is to be appreciated that the chassis 102 and elastic belts 106, 108 may be configured in different ways other than as depicted in FIG. 2. The belt elastic material may be joined to the outer and/or inner layers continuously or intermittently along the interface between the belt elastic material and the inner and/or outer belt layers.

In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168, 172 that extend along non-linear or curved paths that may correspond with the curved contours of the inner lateral edges 107b, 109b.

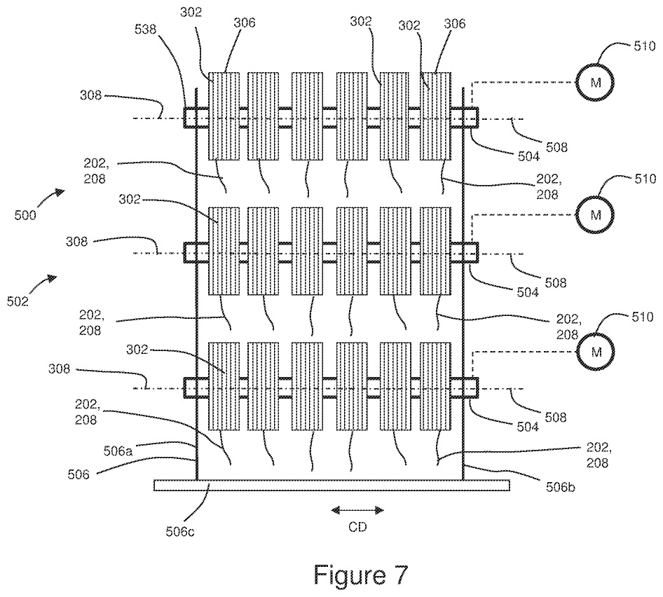

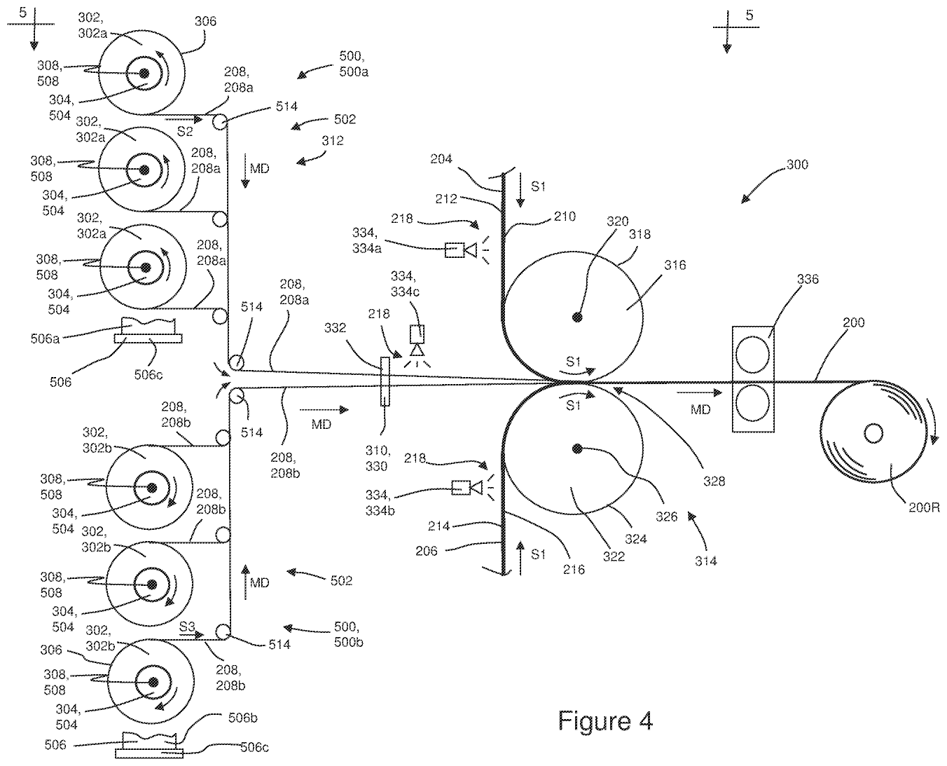

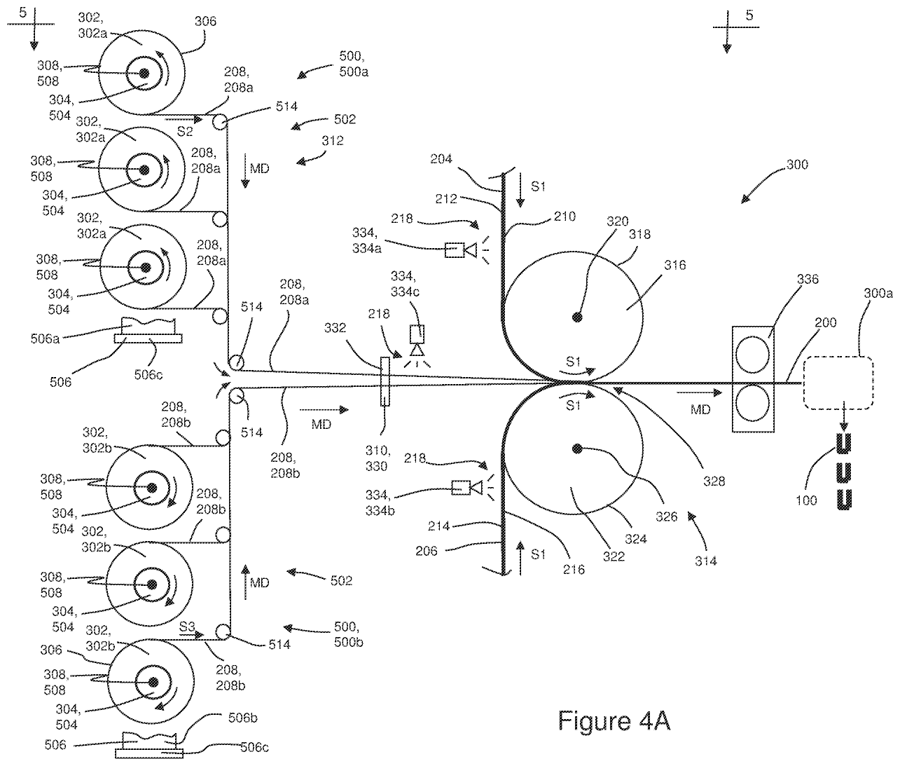

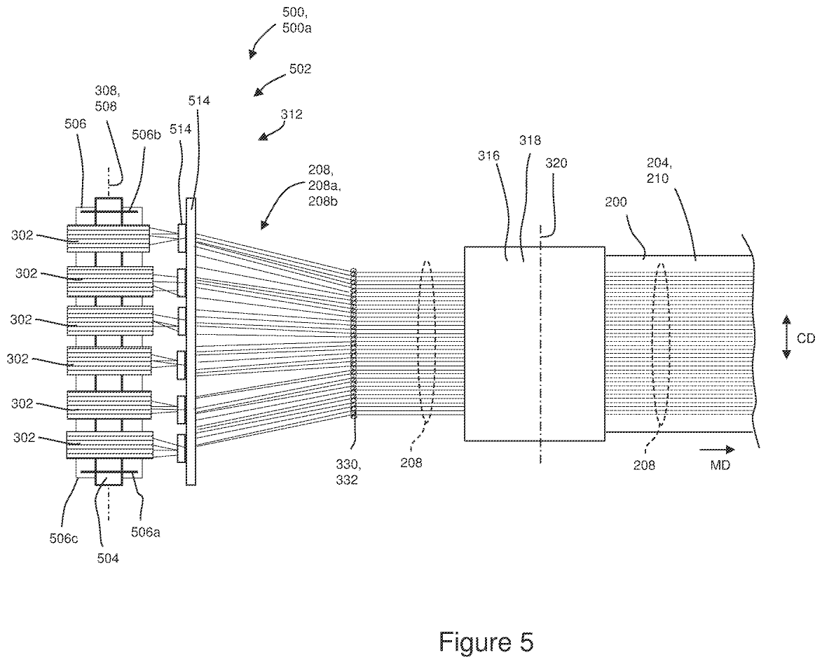

As previously mentioned, apparatuses and methods according to the present disclosure may be utilized to produce elastomeric laminates that may be used to construct various components of diapers, such as elastic belts, leg cuffs, and the like. For example, FIGS. 4 and 5 show schematic views of a converting apparatus 300 adapted to manufacture elastomeric laminates 200. As described in more detail below, the converting apparatus 300 shown in FIGS. 4 and 5 operates to advance a continuous length of elastic material 202, a continuous length of a first substrate 204, and a continuous length of a second substrate 206 along a machine direction MD. It is also to be appreciated that in some configurations, the first substrate and second substrate 204, 206 herein may be defined by two discrete substrates or may be defined by folded portions of a single substrate. The apparatus 300 stretches the elastic material 202 and joins the stretched elastic material 202 with the first and second substrates 204, 206 to produce an elastomeric laminate 200. Although the elastic material 202 is illustrated and referred to herein as strands 208, it is to be appreciated that in some configurations, elastic material 202 may include one or more continuous lengths of elastic strands, ribbons, and/or films.

It is to be appreciated that the elastomeric laminates 200 can be used to construct various types of absorbent article components. It also to be appreciated that the methods and apparatuses herein may be adapted to operate with various types of absorbent article assembly processes, such as disclosed for example in U.S. Patent Publication Nos. 2013/0255861 A1; 2013/0255862 A1; 2013/0255863 A1; 2013/0255864 A1; and 2013/0255865 A1, which are all incorporated by reference herein. For example, the elastomeric laminates 200 may be used as a continuous length of elastomeric belt material that may be converted into the first and second elastic belts 106, 108 discussed above with reference to FIGS. 1A-3B. As such, the elastic material 202 may correspond with the belt elastic material 168 interposed between the outer layer 162 and the inner layer 164, which in turn, may correspond with either the first and/or second substrates 204, 206. In other examples, the elastomeric laminates 200 may be used to construct waistbands and/or side panels in taped diaper configurations. In yet other examples, the elastomeric laminates 200 may be used to construct various types of leg cuff and/or topsheet configurations.

FIGS. 4 and 5 show an example of a converting apparatus 300 that may be configured to assemble elastomeric laminates 200. The apparatus 300 may include a plurality of spools 302 of elastic strands 208. As shown in FIG. 6, each spool 302 may include a single elastic strand 208 wound onto a core 304. The spool 302 may be cylindrically shaped and include an outer circumferential surface 306 defined by the elastic strand 208 wound around the core 304. The spool 302 may also be adapted to rotate about an axis of rotation 308. The core 304 may be cylindrically shaped and the axis of rotation 308 may extend axially through the center of the core 304. With continued reference to FIGS. 4 and 5, the elastic strands 208 are unwound from respective spools 302 by rotating the spools 302 about the cores 304 and/or the axis of rotation 308. The elastic strands 28 advance in a machine direction MD and are combined with the first substrate 204 and the second substrate 206 to form the elastomeric laminate 200.

As shown in FIG. 4, the elastic strands 208 may also advance through a strand guide 310 before being combined with the first substrate 204 and the second substrate 206. As discussed in more detail below, the strand guide 310 spaces or separates neighboring elastic strands 208 from each other at a desired distance in a cross direction CD while being combined with the first substrate 204 and the second substrate 206. The elastic strands 208 may also be stretched in the machine direction MD and combined with the first substrate 204 and the second substrate 206 in the stretched state. As such, tension on the elastomeric laminate 200 may then be reduced to allow the stretched elastic strands 208 to contract and form a gathered elastomeric laminate 200. The gathered elastomeric laminate 200 may be accumulated, such as for example, by being wound onto a roll 200R or being festooned in a container. The accumulated elastomeric laminate 200 may be stored and/or moved to a location for incorporation into an absorbent article assembly process wherein the elastomeric laminate 200 may be converted into an absorbent article component. It is to be appreciated that in some configurations, tension may not be reduced on the elastomeric laminate 200 as the elastomeric laminate is accumulated. As such, the elastomeric laminated 200 may be accumulated under tension on a roll for example, stored, and/or moved to a location for incorporation into an absorbent article assembly process. Thus, tension could be maintained on the elastomeric laminate 200 while being unwound and while being incorporated into an absorbent article assembly process, and such tension can be removed from the elastomeric laminate 200 during the assembly process or after the assembly process is complete.

As shown in FIGS. 4 and 5, the converting apparatus 300 for producing an elastomeric laminate 200 may include a first metering device 312 and a second metering device 314. The first metering device 312 may be configured as an unwinder 500 with one or more spools 302 of elastic strands 208 positioned thereon. During operation, the elastic strands 208 advance in the machine direction MD from the unwinder 500 to the second metering device 314. In addition, the elastic strands 208 may be stretched along the machine direction MD while advancing between the unwinder 500 and the second metering device 314. The stretched elastic strands 208 are also joined with the first substrate 204 and the second substrate 206 at the second metering device 314 to produce an elastomeric laminate 200. It is also to be appreciated that the elastic strands 208 may advance along and/or around one or more guide rollers 514 It is to be appreciated that the elastic strands may be stretched along a continuous path while advancing in the machine direction or may be stretched in various steps that provide multiple increases in elongation while advancing in the machine direction.

As shown in FIG. 4, the second metering device 314 includes: a first roller 316 having an outer circumferential surface 318 and rotates about a first axis of rotation 320, and a second roller 322 having an outer circumferential surface 324 and rotates about a second axis of rotation 326. The first roller 316 and the second roller 322 rotate in opposite directions, and the first roller 316 is adjacent the second roller 322 to define a nip 328 between the first roller 316 and the second roller 322. The first roller 316 may rotate such that the outer circumferential surface 318 has a surface speed S1, and the second roller 322 may rotate such that the outer circumferential surface 324 has the same, or substantially the same, surface speed S1.

As shown in FIG. 4, the first substrate 204 includes a first surface 210 and an opposing second surface 212, and the first substrate 204 advances to the first roller 316. In particular, the first substrate 204 advances at speed S1 to the first roller 316 where the first substrate 204 partially wraps around the outer circumferential surface 318 of the first roller 316 and advances through the nip 328. As such, the first surface 210 of the first substrate 204 travels in the same direction as and in contact with the outer circumferential surface 318 of the first roller 316. In addition, the second substrate 206 includes a first surface 214 and an opposing second surface 216, and the second substrate 206 advances to the second roller 322. In particular, the second substrate 206 advances at speed S1 to the second roller 322 where the second substrate 206 partially wraps around the outer circumferential surface 324 of the second roller 322 and advances through the nip 328. As such, the second surface 216 of the second substrate 206 travels in the same direction as and in contact with the outer circumferential surface 324 of the second roller 322. It is to be appreciated that the first and/or substrates 204, 206 may advance at various speeds S1. In some configurations, the first substrate 204 and/or the second substrate 206 may advance at speed S1 from about 150 meters/minute to about 300 meters/minute, specifically reciting all 1 meter/minute increments within the above-recited range and all ranges formed therein or thereby.

With continued reference to FIGS. 4, 5, and 7, the unwinder 500 may include spools 302 of elastic strands 208 wound thereon, wherein each spool 302 is rotatable about a respective axis of rotation 308. As discussed above, the spools 316 may rotate such that the outer circumferential surface 306 of the spools 302 move at a speed S2. As the spools 302 rotate, the elastic strands 208 unwind from the rotating spools 302 and advance at the speed S2 in the machine direction MD to the nip 328. In some configurations, the speed S2 is less than the speed S1, and as such, the elastic strands 208 are stretched in the machine direction MD. In turn, the stretched elastic strands 208 advance through the nip 328 between the first and second substrates 204, 206 such that the elastic strands 208 are joined with the second surface 212 of the first substrate 204 and the first surface 214 of the second substrate 206 to produce a continuous length of elastomeric laminate 200.

As shown in FIGS. 4 and 8, the elastic strands may advance through a strand guide 310 positioned between the spools 302 and the nip 328. The strand guide 310 may operate to change and/or dictate and/or fix the cross directional CD separation distance between neighboring elastic strands 208 advancing into the nip 328 and in the assembled elastomeric laminate 200. It is to be appreciated that the elastic strands 208 may be separated from each other by various distances in the cross direction CD advancing into the nip 328 and in the assembled elastomeric laminate 200. In some configurations, neighboring elastic strands 208 may be separated from each other by about 0.5 mm to about 4 mm in the cross direction CD, specifically reciting all 0.1 mm increments within the above-recited range and all ranges formed therein or thereby. It is to be appreciated that the strand guide 310 may be configured in various ways. In some configurations, such as shown in FIG. 8, the strand guide 310 may be configured as a comb 330 that may comprise a plurality of tines or reeds 332. In turn, the advancing elastic strands 208 are separated and spaced apart from each other by the tines or reeds 332 in the cross direction CD from each other. In some configurations, the strand guide 310 may include a plurality of rollers that separate and space the elastic strands in the cross direction CD from each other.

As discussed above, it is to be appreciated that the elastomeric laminates 200 assembled herein may include various quantities of elastic strands 208 spaced apart from each other by various distances and may include various decitex values. For example, the elastomeric laminates 200 herein may have various elastic densities, wherein the elastic density may be defined as decitex per elastomeric laminate width. For example, some elastomeric laminates 200 may have an elastic density from about 30 decitex/mm to about 150 decitex/mm, specifically reciting all 1 decitex/mm increments within the above-recited range and all ranges formed therein or thereby. In another example, the elastomeric laminates 200 herein may have various numbers of elastic strands arranged in the cross direction CD per meter of elastomeric laminate cross directional width. For example, some elastomeric laminates 200 may have from about 500 elastic strands/meter of elastomeric laminate width to about 2000 elastic strands/meter of elastomeric laminate width, specifically reciting all 1 elastic strand/meter increments within the above-recited range and all ranges formed therein or thereby.

As shown in FIG. 4, the apparatus 300 may include one or more adhesive applicator devices 334 that may apply adhesive 218 to at least one of the elastic strands 208, the first substrate 204, and the second substrate 206 before being combined to form the elastomeric laminate 200. For example, the first substrate 204 may advance past an adhesive applicator device 334a that applies adhesive 218 to the second surface 212 of the first substrate 204 before advancing to the nip 328. It is to be appreciated that the adhesive 218 may be applied to the first substrate 204 upstream of the first roller 316 and/or while the first substrate 204 is partially wrapped around the outer circumferential surface 318 of the first roller 316. In another example, the second substrate 206 may advance past an adhesive applicator device 334b that applies adhesive 218 to the first surface 214 of the second substrate 206 before advancing to the nip 328. It is to be appreciated that the adhesive 218 may be applied to the second substrate 206 upstream of the second roller 322 and/or while the second substrate 206 is partially wrapped around the outer circumferential surface 324 of the second roller 324. In another example, an adhesive applicator device 334c may be configured to apply adhesive 218 to the elastic strands 208 before and/or while being joined with first substrate 204 and second substrate 206.

It is to be appreciated that the adhesive applicator devices herein 334 be configured in various ways, such as for example, spray nozzles and/or slot coating devices. In some configurations, the adhesive applicator devices 334 may be configured in accordance with the apparatuses and/or methods disclosed in U.S. Pat. Nos. 8,186,296; 9,265,672; 9,248,054; and 9,295,590 and U.S. Patent Publication No. 2014/0148773 A1, all of which are incorporated by reference herein.

As shown in FIG. 4, the apparatus 300 may include a mechanical bonding device 336 that applies the mechanical bonds to the elastomeric laminate 200, such as for example, bonds that may be applied with heat, pressure, and/or ultrasonic devices. Examples of such mechanical bonding devices and methods are disclosed in U.S. Pat. Nos. 4,854,984; 6,291,039; 6,248,195; 8,778,127; and 9,005,392; and U.S. Patent Publication Nos. 2014/0377513 A1; and 2014/0377506 A1, all of which are incorporated by reference herein. It is to be appreciated that the mechanical bonding device 336 may apply mechanical bonds to the elastomeric laminate at or downstream of the nip 328. The mechanical bonding device may apply bonds that bond the first substrate 204, the second substrate 206, and/or elastic strands 208 together and/or may act to trap or immobilize discrete lengths of the contracted elastic strands 208 in the elastomeric laminate 200. It is also to be appreciated that the apparatuses herein may include one of, some of, or all of adhesive applicator devices 334a, 334b, 334c and mechanical bonding device 336 mentioned herein.

It is also to be appreciated that the elastic strands 208 may be bonded with the first substrate 204 and/or second substrate 206 with various methods and apparatuses to create various elastomeric laminates, such as described in U.S. Patent Publication Nos. US20180168878 A1; US20180168877 A1; US20180168880 A1; US20180170027 A1; US20180169964 A1; US20180168879 A1; US20180170026 A1; US20180168889 A1; US20180168874 A1; US20180168875 A1; US20180168890 A1; US20180168887 A1; US20180168892 A1; US20180168876 A1; US20180168891 A1; US20190070042 A1; and US20190070041 A1 and combinations thereof, all of which are incorporated herein by reference.

It is to be appreciated that different components may be used to construct the elastomeric laminates 200 in accordance with the methods and apparatuses herein. For example, the first and/or second substrates 204, 206 may include nonwovens and/or films. In addition, the elastic strands 208 may be configured in various ways and may have various decitex values. In some configurations, the elastic strands 208 may be configured with decitex values ranging from about 10 decitex to about 500 decitex, specifically reciting all 1 decitex increments within the above-recited range and all ranges formed therein or thereby.

As shown in FIGS. 4 and 5, the elastomeric laminate 200 may advance from the nip 328 and may be accumulated, such as for example, by being wound onto a roll 200R or being festooned in a container. It is to be appreciated that the elastomeric laminate 200 may be wound onto a roll 200R in a fully stretched, partially stretched, or fully relaxed state. The accumulated elastomeric laminate 200 may be stored and/or moved to a location for incorporation into an absorbent article assembly process, wherein the elastomeric laminate 200 may be converted into an absorbent article component, such as discussed above. As such, the accumulated elastomeric laminate 200 may be unwound from a roll 200R (or drawn from a container) and incorporated into an absorbent article assembly line. It is to be appreciated that the apparatus 300 may be configured to assemble elastomeric laminates 200 that may be cut along the machine direction MD to define separate lanes of elastic of individual elastomeric laminates 200. In some configurations, the elastomeric laminate may be cut into separate lanes of individual elastomeric laminates 200 before wound onto respective rolls 200R. In some configurations, the elastomeric laminate may be cut into separate lanes of individual elastomeric laminates 200 as the elastomeric laminate is unwound from a roll 200R.

It is to be appreciated that in some configurations, the elastomeric laminate 200 may advance from the nip 328 and may be incorporated directly into an absorbent article assembly process without first being accumulated. For example, FIG. 4A shows the elastomeric laminate 200 advancing from the nip 328 directly into an absorbent article assembly line 300a, generically represented by rectangle in dashed lines, without first being accumulated. The absorbent article assembly may be configured to convert the elastic laminate 200 along with additional components to assembly absorbent articles 100, such as diapers. It is to be appreciated that the unwinders 500 may be located in various positions relative the absorbent article assembly line 300a. For example, in some configurations, the unwinders 500 may be located on a mezzanine adjacent and/or above the absorbent article assembly line 300a.

As previously mentioned, the apparatus 300 may include an unwinder 500 including spools 302 of elastic strands 208. It is to be appreciated the unwinder 500 may be configured with various quantities of spools 302 of elastic strands 208. Although FIG. 7 shows eighteen spools 302 positioned on the unwinder 500, and correspondingly, eighteen elastic strands 208 that may advance from the unwinder 500, it is to be appreciated that the unwinders 500 herein may be configured with more or less than eighteen spools 302 and more or less than eighteen elastic strands 208 advancing from the unwinder 500. In some configurations, the unwinders 500 herein may include from 1 to about 3000 spools 302 positioned thereon, and thus, may have from 1 to about 3000 elastic strands 208 advancing therefrom, specifically reciting all 1 spool and strand increments within the above-recited range and all ranges formed therein or thereby. In turn, the elastomeric laminates 200 herein may include from 1 to about 3000 elastic strands 208 spaced apart from each other in the cross direction CD, specifically reciting all 1 elastic strand increments within the above-recited range and all ranges formed therein or thereby.

It is also to be appreciated the unwinder 500 may be configured in various ways. For example, the unwinder 500 may be configured as a creel 502 adapted to support one or more spools 302 of elastic strands 208. FIG. 7 shows an example of an unwinder 500 that may include one or more mandrels 504 connected with a frame 506. It is to be appreciated that the frame 506 may be configured in various ways. For example, the frame 506 may include a first side 506a and a second side 506b connected with a base 506c. For the purposes of clarity, the first side 506a and the second side 506b are illustrated as being partially cut-away in FIG. 4. With continued reference to FIGS. 4, 5, and 7, the mandrels 504 may be rotatably connected with the frame 506 and may be adapted to rotate about a mandrel rotation axis 508. It is to be appreciated that the mandrels 504 may be oriented in various ways. For example, mandrels 504 may be horizontally or vertically oriented.

As shown in FIGS. 4, 5, 6, and 7, one or more spools 302 may be positioned on and supported by mandrels 504 of the unwinder 500. In some configurations, the cores 304 of one or more spools 302 may be adapted to receive and/or connect with the mandrel 504. As such, the spools 302 and the mandrel 504 may be adapted to rotate together. In some configurations, the mandrel 504 may be configured to drive and cause rotation of the spools 302. For example, FIG. 7 shows the mandrel 504 connected with a rotation driver 510, such as a motor or a servo motor, to drive and control the rotation of the mandrel 504. During operation, each spool 302 and the mandrel 504 are rotated in the same direction. An elastic strand 208 advances from the rotating spool 302 to downstream assembly operations, such as described herein. The unwinder 500 may also be configured such that the elastic strands 208 advance from the spools 302 at a speed S2 as described above. As elastic strands 208 are drawn from the rotating spools 302 supported on the mandrel 504, the outer diameter of the spools 302 become smaller. In turn, as the outer diameter of the spools 302 become smaller, the rotational speed of the mandrel 504 and spools 302 may need to increase in order to maintain a constant speed S2 of the elastic strands 208 advancing from the spools 302. As such, the apparatus 300 herein may include a sensor that detects the diameter of the spools 302, wherein feedback from the sensor can be used to control the speed of the rotation driver 510 and mandrel 504 to maintain a constant speed S2. In some configurations, the sensor may be configured to detect the tension in the elastic strands 208, wherein feedback from the sensor can be used to control the speed of the rotation driver 510 and/or mandrel 504 and/or mandrel 504 to maintain a desired tension in the strand 208.

As previously mentioned, one or more spools 302 may be positioned on and supported by the mandrel 504. And as shown in FIG. 7, the unwinder 500 may include one more mandrels 504 rotatably connected with a frame 506. It is to be appreciated that rotation drivers 510 may be directly connected with one or more mandrels 504 or indirectly connected with mandrels 504, such as through a transmission device, such as gear, pulley, chain, and/or belt arrangements. It is also to be appreciated that the mandrels 504 may be adapted to rotate independently of each other. In some configurations, the mandrels 504 may be rotationally connected with each other through a transmission device. It is to be appreciated that unwinder 500 may be connected with various arrangements of rotation drivers 510 adapted to rotate the mandrels 504 and/or spools 302 at the same or different speeds. For example, a plurality of mandrels 504 on the unwinder 500 may be connected with a single rotation driver 510 that may rotate the plurality of mandrels 504 and spools 302 thereon at the same speed. In another example, a plurality of mandrels 504 on the unwinder 500 may be connected with a single rotation driver 510 through a transmission device, and as such, may be configured to the drive the mandrels 504 and spools 302 thereon at the same or different speeds. In yet another example, a plurality of rotation drivers 510 may be configured to drive respective mandrels 504, each mandrel 504 having one or more spools 302 thereon. As such, the rotation drivers 510 may be configured to rotate respective mandrels 504 and spools 302 at the same or different speeds. In some configurations, spools 302 may be rotatably supported by the unwinder 500 without being driven, and as such, may be adapted to rotate as a result of respective elastic strands 208 being drawn therefrom.

It is also to be appreciated that one or more unwinders 500 and spools 302 of elastics 208 positioned thereon may be arranged along the cross direction CD of a converting process and/or arranged along a machine direction MD in various different portions of a converting process. For example, FIGS. 4 and 5 show an arrangement that includes a first unwinder 500a with first spools 302a of elastic strands 208a and a second unwinder 500b with second spools 302b of elastic strands 208b. The first and second elastic strands 208a, 208b may advance from the respective first and second unwinders 500a, 500b to be incorporated into the elastomeric laminate 200.

It is to be appreciated that the apparatuses and processes may be configured such that elastic strands 208 may be advanced from the unwinders 500 and directly to the assembly process without having to touch additional machine components, such as for example, guide rollers 514. It is also to be appreciated that in some configurations, elastic strands 208 may be advanced from the unwinders 500 and may be redirected and/or otherwise touched by and/or redirected by machine components, such as for example guide rollers 514, before advancing to the assembly process. Thus, it is to be appreciated that the first and/or second unwinders 500a, 500b and associated spools 302a, 302b may be arranged and/or oriented such that the rotation axes 508 of the mandrels 504 and/or rotation axes 308 of spools 302 may be parallel, perpendicular, or otherwise angularly offset with respect to the machine direction advancement of the elastomeric laminate 200 and/or the substrates 204, 206.

During assembly operations, spools 302 of elastic strands 208 may become depleted and may require replacement. As such, empty or nearly depleted individual spools 302 may be replaced with fresh spools of elastic strands 208. In some configurations, once spools 302 are empty or nearly depleted of elastic strands 208, replacement elastic strands 208 can be introduced into the assembly operation as replacements for the original elastic strands 208 without having to stop the assembly operation. For example, replacement elastic strands 208 can be spliced to elastic strands 208 on depleted spools. Such replacement and splicing operations may be accomplished on an individual spool basis or may be accomplished by splicing a plurality of spools at the same time.

In some configurations, empty or depleted spools 302 on an unwinder 500 can be replaced with an unwinder with replenished spools 302 with elastic strands 208 wound thereon positioned to replace elastic strands 208 once depleted from the spools 302 on the unwinder 500. Subsequently, advancement of the elastic strands from the depleted spools to the downstream assembly operations may be discontinued. As such, the elastomeric laminate assembly process may continue uninterrupted while replacing elastic strands 208 unwound from the depleted spools with elastic strands 208 unwound from the replacement spools. It is to be appreciated that various types of splicing operations may be utilized, such as disclosed for example, in U.S. Patent Publication Nos. 2018/0168878 A1 and 2018/0170026 A1, both of which are incorporated herein by reference.

As previously mentioned, the apparatuses 300 herein may be configured to a plurality of unwinders 500, and such arrangements may be utilized in splicing operations. For example, during assembly operations utilizing elastic strands 208a from first spools 302a on a first unwinder 500a, splices may be prepared with elastic strands 208b from second spools 302b on a second unwinder 500b. In turn, assembly operations may be temporarily stopped and the splicing operation could be executed manually during a relatively short period of time. With such a splicing operation, all the spools 302 may be configured to include relatively equal quantities of elastic strands, which may be based on assumed consumption rate, considering that linear meters of elastic consumption may be different on some spools based on different elastic strand tensions. It is also to be appreciated that some splicing operations may be automated.

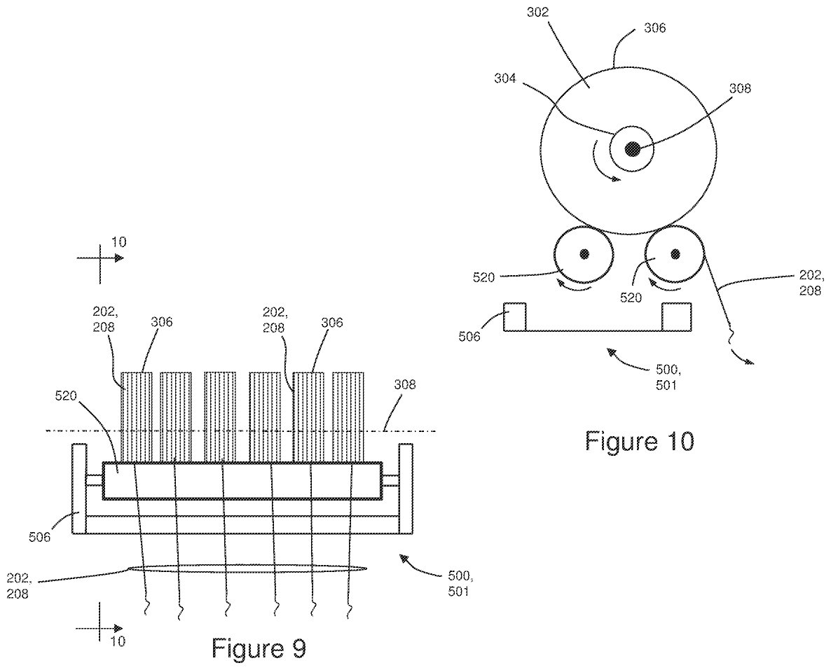

Although FIGS. 4, 5, and 7 illustrate unwinders 500 configured as mandrel driven unwinders, it is to be appreciated that the unwinders 500 herein may be configured in different ways. For example, the unwinders 500 may also be configured as surface driven unwinders 501, wherein the spools 302 may be driven by one or more rolls 520 in contact with the outer circumferential surfaces 306 of the spools 302, such as shown in FIGS. 9 and 10 and as disclosed in U.S. Patent Publication No. 2018/0170026 A1, which is incorporated by reference herein. It is also to be appreciated that surface driven unwinders 501 may also be configured to operate with spools 302 arranged in various ways, such as horizontal or vertical orientations. Different arrangements of spools 302 on unwinders 500 may be desirable for various reasons, such as for example, based on limited available space considerations. For example, surface driven unwinders may be configured to unwind elastic strands 208 from vertically arranged or stacked spools 302 with vertically oriented rotational axes 308, such as for example, available from Karl Mayer. Corporation.

In addition, the apparatus 300 may be configured to assemble elastomeric laminates 200 with elastic strands 208 unwound from more than one unwinder 500 in combination with elastic strands supplied from various other types of elastic unwinder configurations, such as an overend unwinder and/or beams (also referred to as warp beams), such as disclosed in U.S. Pat. Nos. 6,676,054; 7,878,447; 7,905,446; 9,156,648; 4,525,905; 5,060,881; and 5,775,380; and U.S. Patent Publication No. 2004/0219854 A1, all of which are incorporated by reference herein. Additional examples of elastics and associated handling equipment are available from Karl Mayer Corporation.

As previously mentioned, the elastic strands 208 may include various types of spin finish, also referred herein as yarn finish, configured as coating on the elastic strands 208 that may be intended to help prevent the elastic strands from adhering to themselves, each other, and/or downstream handling equipment. In some configurations, a spin finish may include various types of oils and other components, such as disclosed for example in U.S. Pat. Nos. 8,377,554; 8,093,161; and 6,821,301, all of which are incorporated by reference herein. In some configurations, a spin finish may include various types of silicone oils, such as for example, polydimethylsiloxane. In some configurations, a spin finish may include various types of mineral oils, including hydrogenated paraffinic and napthenic oils. In some configurations, the molecular weight of an oil may be adjusted to optimize adhesion properties of the elastic strands depending on the process configuration in which the elastic strands may be used. In some configurations, a spin finish may include various types of fatty amides, erucamide, behenamide, and oleamide.

It is to be appreciated that the elastic strands 208 may not include any spin finish or may require relatively low amounts of spin finish. As such, relatively low amounts of adhesive 218 may be required to adequately adhere stretched elastic strands 208 without spin finish or relatively low quantities of spin finish to substrates than would otherwise be required for elastic strands 208 with relatively large amounts of spin finish. In turn, relatively smaller amounts of adhesives 218 required to bond elastic strands 208 to the substrates may have a positive impact on aspects of a resulting product, such as with respect to costs, functionality, and aesthetics.

In some configurations, elastic strands 208 having relatively larger decitex values and/or elastic strands 208 wound onto spools 302 with relatively low tensions may not require any spin finish or may require relatively lower amounts of spin finish to help prevent the elastic strands 208 from adhering to themselves. In some configurations, spin finish may be applied to the elastic strands 208 before, during, and/or after being wound onto respective spools 302. It is also to be appreciated that the amount of spin finish applied to elastic strands may be optimized depending on the process configuration in which the elastic strands 208 may be used. For example, in process configurations wherein elastic strands have limited contact or do not contact downstream handling equipment, such as idlers, the amount of spin finish may be selected to help prevent the elastic strands 208 from adhering to themselves and/or each other while wound on a spool 302 without regard to whether elastic strands 208 would adhere to downstream handling equipment.

As such, it is to be appreciated that the elastic strands 208 herein may include various amounts of spin finish that may be expressed in various ways. For example, a quantity of 10 grams of spin finish per 1 kilogram of elastic strand may be expressed as 1% spin finish. In some configurations, an elastic strand may include about 0.1% spin finish. In some configurations, a strand may include from about 0.01% to about 10% spin finish, specifically reciting all 0.01% increments within the above-recited range and all ranges formed therein or thereby. It is also to be appreciated that the methods and apparatuses herein may also be configured to remove some or all the spin finish from the elastic strands 208. Examples of spin finish removal processes and apparatuses are disclosed in U.S. Patent Publication No. 2018/0168877, which is incorporated by reference herein.

It is to be appreciated that the apparatuses 300 herein may be configured in various ways with various features described herein to assemble elastomeric laminates 200 having various stretch characteristics. For example, when the elastomeric laminate 200 is elongated, some elastic strands 208 may exert contraction forces in the machine direction MD that are different from contraction forces exerted by other elastic strands 208. Such differential stretch characteristics can be achieved by stretching some elastic strands 208 more or less than other elastic strands 208 before joining the elastic strands with the first and second substrates 204, 206. As discussed above, the spools 302 of elastic strands 208 may be unwound from one or more unwinders 500 at different speeds from each other, and as such, the elastic strands 208 may be stretched more or less than each when combined with the first and second substrates. For example, as previously discussed, the first substrate 204 and the second substrate 206 may each advance at a speed S1. In some configurations, the first elastic strands 208a may advance from first spools 302a at speed S2 that is less than the speed S1, and second elastic strands 208b may advance from second spools 302b at the speed S3 that is less than the speed S1. As such, the first elastic strands 208a and the second elastic strands 208b are stretched in the machine direction MD when combined with the first and second substrates 204, 206. In addition, the speed S2 may be less than or greater than the speed S3. Thus, the first elastic strands 208a may be stretched more or less than the second elastic strands 208b when combined with the first and second substrates 204, 206.

As discussed herein, the elastic strands 208 may be pre-strained prior to joining the elastic strands 208 to the first or second substrate layers 204, 206. In some configurations, the elastic strands 208 may be pre-strained from about 75% to about 300%, specifically reciting all 1% increments within the above-recited range and all ranges formed therein or thereby. In some configurations, the elastic strands 208 may be pre-strained from about 80% to about 250%, specifically reciting all 1% increments within the above-recited range and all ranges formed therein or thereby. Pre-strain refers to the strain imposed on an elastic or elastomeric material prior to combining it with another element of the elastomeric laminate or the absorbent article. Pre-strain is determined by the following equation: Pre-strain=((extended length of the elastic-relaxed length of the elastic)/relaxed length of the elastic)*100.

It is also to be appreciated that the elastic strands 208 may have various different material constructions and/or decitex values to create elastomeric laminates 200 having different stretch characteristics in different regions. In some configurations, the spools 302 of elastic strands 208 having different decitex values may be positioned on and advanced from one or more unwinders 500. In some configurations, the elastomeric laminate 200 may have regions where the elastic strands 208 are spaced relatively close to one another in the cross direction CD and other regions where the elastic strands 208 are spaced relatively far apart from each other in the cross direction CD to create different stretch characteristics in different regions. In some configurations, the elastic strands 208 may be supplied on the spool 302 in a stretched state, and as such, may not require additional stretching (or may require relatively less additional stretching) before being combined with the first substrate 204 and/or the second substrate 206. In some configurations, differential stretch characteristics in an elastomeric laminate 200 may be created by bonding another substrate and/or elastomeric laminate and/or an elastic film to a particular region of an elastomeric laminate. In some configurations, differential stretch characteristics in an elastomeric laminate 200 may be created by folding a portion of an elastomeric laminate onto itself in a particular region of the elastomeric laminate.

In some configurations, the elastic strands 208 may be joined with the first and second substrates 204, 206 such that the elastomeric laminate 200 may have different stretch characteristics in different regions along the cross direction CD, such as disclosed in U.S. Patent Publication Nos. 2006/0094319A1; US2006/0032578A1; 2018/0168878 A1; 2018/0168877 A1; 2018/0168880 A1; 2018/0170027 A1; US20180169964 A1; US20180168879 A1; 20180170026 A1; 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0070042 A1; and 2019/0070041 A1, which are all incorporated by reference herein. In some configurations, the elastomeric laminate 200 may include different tension zones that may help make some web handling operations less cumbersome, such as disclosed in U.S. Patent Publication No. 2002/0009940 A1, which is incorporated by reference herein.

It is to be appreciated that various operational abnormalities may result while elastic strands 208 are advancing from spools 302 during assembly operations disclosed herein. For example, breakouts may occur during assembly operations, wherein one or more elastic strands 208 unintentionally breaks while advancing from the spool 302 during assembly of the elastomeric laminate 200. As such, the methods and apparatuses herein may include various devices to help isolate broken elastic strands, such as disclosed in U.S. Patent Publication Nos. 2014/0209652 A1 and 2014/0224855 A1, which are incorporated by reference herein. In some instances, the methods and apparatuses may include a snare member adjacent spools 302, strand guides 310, and/or other assembly components to help isolate broken elastics strands, such as disclosed in U.S. Patent Publication No. 2015/0090393 A1, which is incorporated by reference herein. The apparatuses and methods herein may also be configured with a two-step elastic strand straining process, wherein the elastic strands 208 advance from the spool 302 and through a nip and drive roll before advancing in the machine direction to be combined with the first and second substrates 204, 206. Such a nip and drive roll arrangement may help isolate broken elastic strands from other elastic strands and/or handling equipment. The apparatuses and methods herein may also be configured with devices and other arrangements to help automatically rethread broken elastic strands 208, such as disclosed in U.S. Patent Publication Nos. 2013/0199707 A1 and 2013/0199696 A1, which are incorporated by reference herein. In some configurations, spools 302 may be wound with elastic strands 208 having pieces of tape extending across the strands, wherein the tape pieces are intermittently spaced apart along the machine direction. As such, the tape pieces may help in locating the end of a broken strand in the event of a breakout.

It is also to be appreciated that the assembly operations may be configured to help reduce the chances of elastic strand breakouts. For example, the first substrate 204 and/or the second substrate 206 may be configured as pre-corrugated nonwovens. As such, the elastic strands 208 may be bonded with the substrates 204, 206 at relatively lower strains than required in the final assembled elastomeric laminate 200. Thus, relatively lower strains in the stretched elastic strands 208 may reduce the likelihood that such elastic strands break during assembly operations.

It is also to be appreciated that the elastomeric laminate assembly operations herein may also be performed in conjunction with other operations.

In some configurations, the elastomeric laminates 200 assembled with the methods and apparatuses herein may be subjected to various other manufacturing transformations before or after being accumulated. As discussed above, a continuous elastomeric laminate 200 may advance to a slitting operation, wherein the elastomeric laminate 200 is slit and separated along the machine direction MD into lanes, such as for example, a first continuous elastomeric laminate and a second continuous elastomeric laminate. It is to be appreciated that the elastomeric laminate 200 may be slit with a shear slitting operation or a crush slit operation. In a crush slit operation, the first substrate 204 and the second substrate 206 may be bonded together during the slitting operation. In some operations, the first and second substrates 204, 206 of an elastomeric laminate 200 may be bonded together along edges of the elastomeric laminate 200. For example, in some operations, edges of the first substrate 204 may be folded over opposing edge portions of the second substrate 206 to create sealed edges of the elastomeric laminate 200. It is to be appreciated that heat, pressure, adhesive, and/or ultrasonic bonding processes may be used to fixate such folded portions of the substrates. In some configurations, the locations of elastic strands 208 relative to side edges of elastomeric laminates 200 may be adjusted to change corrugation patterns along the side edges in desired manners. The elastomeric laminates 200 herein may be subject to additional operations to help provide aesthetic benefits, such as relatively more homogenous and/or consistent widths along the machine direction. In some configurations, edges of elastomeric laminates 200 may be trimmed to help improve aesthetics by providing relatively smooth and/or finished edges. In some configurations, the elastomeric laminates 200 may be subject to cross directional spreading operations that may be executed after the elastomeric laminate has at least partially relaxed.

In some configurations, the first substrate 204 and/or the second substrate 206 may be subjected to aperturing processes during assembly operations of the elastomeric laminate 200. And in some configurations, the assembled elastomeric laminate 200 may be subjected to aperturing processes before or after being accumulated. It is to be appreciated that various different types of aperturing processes and operational configurations may be used, such as disclosed, for example, in U.S. Provisional Patent Application No. 62/874,600, which is incorporated by reference herein. It is also to be appreciated that the first substrate 204, the second substrate 206, and/or the assembled elastomeric laminate 200 may be subjected to various other forming processes, such as embossing and others, such as disclosed, for example, in U.S. Patent Publication Nos. 2018/0228666 A1; 2018/0228656 A1; 2018/0228668 A1; 2019/0183689 A1; and 2018/0228669 A1, which are all incorporated by reference.

In some configurations, the first substrate 204 and/or the second substrate 206 may be subjected to printing operations during assembly operations of the elastomeric laminate 200. And in some configurations, the assembled elastomeric laminate 200 may be subjected to printing processes before or after being accumulated. For example, print stations may be configured to print the first surface 210 and/or the second surface 212 of the first substrate 204 and/or to print the first surface 214 and/or the second surface 216 of the second substrate 206 before being combined to form the elastic laminate 200. In another example, print stations may be configured to print the first substrate 204 and/or to print the second substrate 206 after being combined to form the elastic laminate 200. It is to be appreciated that the printing stations may be configured in various ways and may include various types of printing accessories. For example, the printing stations may be capable of printing ink on substrate materials to form graphics by various printing methods, such as flexographic printing, rotogravure printing, screen-printing, inkjet printing, and the like. In some configurations, one or more lasers may be provided to create laser induced graphics on either or both the first substrate 204 and the second substrate 206.

As used herein, the term “graphic” refers to images or designs that are constituted by a figure (e.g., a line(s)), a symbol or character, a color difference or transition of at least two colors, or the like. A graphic may include an aesthetic image or design that can provide certain benefit(s) when viewed. A graphic may be in the form of a photographic image. A graphic may also be in the form of a 1-dimensional (1-D) or 2-dimensional (2-D) bar code or a quick response (QR) bar code. A graphic design is determined by, for example, the color(s) used in the graphic (individual pure ink or spot colors as well as built process colors), the sizes of the entire graphic (or components of the graphic), the positions of the graphic (or components of the graphic), the movements of the graphic (or components of the graphic), the geometrical shapes of the graphic (or components of the graphics), the number of colors in the graphic, the variations of the color combinations in the graphic, the number of graphics printed, the disappearance of color(s) in the graphic, and the contents of text messages in the graphic.

It is to be appreciated that a control system and/or an inspection system may be utilized to control various aspects of the elastomeric laminate assembly operations discussed herein. For example, as previously mentioned, the unwinders 500 may be connected with one or more motors, such as servo motors, to drive and control the rotation of the spools 302. As such, a control system may operate to control the acceleration and/or deceleration of the spools 302 during the assembly operations and/or splicing operations to achieve and/or maintain the desired tension in the elastic strands 208. In some configurations, the elastic strands 208 may be advanced from the unwinders 500 through a series of dancer rolls to help maintain desired tensions in the elastic strands 208 during assembly and/or splicing operations.

As previously mentioned, the elastomeric laminate 200 may also be subject to additional converting processes after being accumulated. For example, such additional converting processes may incorporate the elastomeric laminate 200 into discrete absorbent articles 100. As such, in some embodiments, an inspection system may be configured to detect and/or track a defective length of the elastomeric laminate 200. For example, a defective length of the elastomeric laminate 200 may include areas where substrates 204, 206 and/or elastic strands 208 have been spliced. In another example, a defective length of the elastomeric laminate 200 may include areas with missing elastic strands 208 and/or broken elastic strands 208. The inspection system may also correlate inspection results and measurements from a defective length of the elastomeric laminate 200 unwound from a roll 200R with absorbent articles 100 made therefrom. In turn, the inspection system may be used to control a reject system on a converting process of absorbent articles, wherein absorbent articles manufactured with portions of the defective length of elastomeric laminate 200 are rejected. In some configurations, defective articles may be subject to the rejection system and removed from the assembly process. Absorbent articles 100 that are not deemed to be defective may be subject to further processing steps, such as folding and packaging.

In some configurations, an inspection system may be configured to detect broken elastic strands 208 advancing from spools 302.

Upon detection of a broken elastic strand, the inspection system may activate a splicing operation, such as described above, to place a replacement spool into service. It is to be appreciated that such an inspection system may be configured in various ways, such as disclosed in U.S. Patent Publication No. 2013/0199696 A1, which is incorporated by reference.

In some configurations, the inspection system may stop elastomeric laminate assembly operations upon detection of one or more broken elastic strands. In some configurations, the elastomeric laminate assembly operations may continue after detection of one or more broken elastic strands or at least until a quantity of the broken elastic strands reaches a specified limit. For example, the inspection system may be configured to detect a broken elastic strand 208 advancing from a spool 302 and may continue the elastomeric laminate assembly operations until a ratio of a number of broken elastic strands to a number of unbroken elastic strands is greater than a specified limit. For example, elastomeric laminate assembly operations may be stopped when the ratio of the number of broken elastic strands to the number of unbroken elastic strands is equal to or greater than 1:100.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling an elastomeric laminate, the method comprising steps of:
providing first spools, each first spool comprising a single first elastic strand;
unwinding the first elastic strands from the first spools;
spacing neighboring first elastic strands at a first distance from each other in a cross direction by advancing the first elastic strands in a machine direction through a strand guide;
stretching the first elastic strands in the machine direction;
combining the first elastic strands with a first substrate and a second substrate to form an elastomeric laminate; and
accumulating the elastomeric laminate.

2. The method of claim 1, further comprising steps of:
reducing tension on the elastomeric laminate to allow the stretched first elastic strands to contract and form a gathered elastomeric laminate; and
accumulating the gathered elastomeric laminate.

3. The method of claim 2, further comprising steps of:
stretching the gathered elastomeric laminate; and
converting the stretched gathered elastomeric laminate into an absorbent article component.

4. The method of claim 3, wherein the absorbent article component comprises an elastic belt.

5. The method of claim 3, further comprising a step of unwinding the gathered elastomeric laminate from a roll.

6. The method of claim 1, further comprising steps of:
maintaining tension on the elastomeric laminate to prevent the stretched elastic strands from contracting; and
accumulating the elastomeric laminate while under tension.

7. The method of claim 1, wherein the step of unwinding the first elastic strands further comprises rotating the first spools.

8. The method of claim 1, further comprising steps of:
providing a substrate; and
folding the substrate to define the first substrate and the second substrate.

9. The method of claim 1, further comprising a step of slitting the elastomeric laminate into a plurality of lanes.

10. The method of claim 1, wherein the first elastic strands do not comprise a spin finish.

11. The method of claim 1, wherein the step of accumulating further comprises winding the elastomeric laminate into a roll.

12. The method of claim 1, wherein the step of accumulating further comprises festooning the elastomeric laminate into a container.

13. The method of claim 1, wherein the step of providing first spools further comprises providing from about 100 to about 3000 first spools.

14. The method of claim 1, wherein the first distance is from about 0.5 mm to about 2 mm.

15. The method of claim 1, further comprising steps of:
providing second spools, each second spool comprising a single second elastic strand;
unwinding the second elastic strands from the second spools by rotating the second spools;
spacing neighboring second elastic strands at a second distance from each other in the cross direction;
stretching the second elastic strands in the machine direction; and
combining the second elastic strands with the first substrate and the second substrate.

16. The method of claim 15, wherein the first distance is different from the second distance.

17. The method of claim 15, wherein the first elastic strands comprise a first decitex and the second elastic strands comprise a second decitex, wherein the first decitex and the second decitex are not equal.

18. The method of claim 15, wherein the elastomeric laminate comprises a first region having a first stretch characteristic defined by the first elastic strands and a second region having a second stretch characteristic defined by the second elastic strands, wherein the first stretch characteristic is different from the second stretch characteristic.

19. The method of claim 15, wherein the second elastic strands are stretched more than the first elastic strands.

20. The method of claim 1, wherein the step of combining further comprises applying adhesive to at least one of the first elastic strands, the first substrate, and the second substrate.

21. The method of claim 1, wherein the step of combining further comprises mechanically bonding the first substrate and the second substrate together.

22. The method of claim 1, further comprising steps of:
detecting one or more broken first elastic strands; and
discontinuing a step of combining unbroken first elastic strands with the first substrate and the second substrate to form the elastomeric laminate when a ratio of a number of broken first elastic strands to a number of unbroken first elastic strands is greater than a limit.

23. The method of claim 1, further comprising steps of:
advancing the first substrate to a printing station;
advancing the printed first substrate from the printing station to a nip; and
combining the first elastic strands with the first printed substrate and the second substrate at the nip.

24. A method for assembling an elastomeric laminate, the method comprising steps of:
providing first spools, each first spool comprising a single first elastic strand;
providing second spools, each second spool comprising a single second elastic strand;
unwinding first elastic strands from first spools and unwinding second elastic strands from second spools;
stretching the first and second elastic strands in the machine direction, wherein the first elastic strands are stretched more than the second elastic strands by rotating first spools and the second spools at different speeds;
spacing neighboring first elastic strands at a first distance from each other in a cross direction by advancing the first elastic strands through reeds;
spacing neighboring second elastic strands at a second distance from each other in the cross direction by advancing the second elastic strands through reeds;
combining the first and second elastic strands with a first substrate and a second substrate to form an elastomeric laminate; and
accumulating the elastomeric laminate.

25. The method of claim 24, further comprising steps of:
reducing tension on the elastomeric laminate to allow the stretched first and second elastic strands to contract and form a gathered elastomeric laminate; and
accumulating the gathered elastomeric laminate.

26. The method of claim 24, further comprising steps of:
maintaining tension on the elastomeric laminate to prevent the stretched elastic strands from contracting; and
accumulating the elastomeric laminate while under tension.

27. The method of claim 24, wherein the first distance is different from the second distance.

28. A method for assembling an elastomeric laminate, the method comprising steps of:
providing first spools, each first spool comprising a single first elastic strand, wherein the first elastic strands comprise a first decitex;
providing second spools, each second spool comprising a single second elastic strand, wherein the second elastic strands comprise a second decitex that is not equal to the first decitex;
unwinding first elastic strands from first spools and unwinding second elastic strands from second spools by rotating the first and second spools;
stretching the first and second elastic strands in a machine direction;
spacing neighboring first elastic strands at a first distance from each other in a cross direction;
spacing neighboring second elastic strands at a second distance from each other in the cross direction;
combining the first and second elastic strands with a first substrate and a second substrate to form an elastomeric laminate;
reducing tension on the elastomeric laminate to allow the stretched first and second elastic strands to contract and form a gathered elastomeric laminate; and
accumulating the gathered elastomeric laminate.

29. A method for assembling an elastomeric laminate, the method comprising steps of:
providing a first unwinder;
positioning a first plurality of first spools on the first unwinder, each first spool comprising a single first elastic strand;
unwinding the first elastic strands from the first spools by rotating the first spools on the first unwinder;
spacing neighboring first elastic strands from each other in a cross direction by advancing the first elastic strands in a machine direction through a strand guide;
stretching the first elastic strands in the machine direction;
combining the first elastic strands with a first substrate and a second substrate to form an elastomeric laminate; and
maintaining tension on the elastomeric laminate to prevent the stretched first elastic strands from contracting.

30. The method of claim 29, wherein the first plurality of spools comprises at least 100 spools.

31. The method of claim 29, further comprising a step of accumulating the elastomeric laminate while under tension.

32. The method of claim 29, further comprising steps of:
converting the elastomeric laminate under tension into an absorbent article component; and
removing tension on the absorbent article component to allow the stretched elastic strands to contract and form a gathered elastomeric laminate.

33. The method of claim 29, further comprising steps of:
providing a second unwinder;
positioning a second plurality of second spools on the second unwinder, each second spool comprising a single second elastic strand;
discontinuing unwinding the first elastic strands from the first spools on the first unwinder;
unwinding the second elastic strands from the second spools by rotating the second spools on the second unwinder;
stretching the second elastic strands in the machine direction; and
combining the second elastic strands with the first substrate and the second substrate to form the elastomeric laminate.

34. The method of claim 33, wherein the second plurality of second spools comprises at least 100 spools.

35. The method of claim 29, wherein the each of the first plurality of spools comprises an axis of rotation, and wherein the step of positioning the first plurality of first spools on the first unwinder further comprises vertically orienting the axis of rotation of each of the first plurality of spools.
